(19) United States
(12) Patent Application Publication
(10) Pub. No.: US 2025/0344702 A1
(43) Pub. Date: Nov. 13, 2025

(54) Sporulated Microbial Formulations

(71) Applicant: Rosen's, Inc., Liberty, MO (US)

(72) Inventors: Matthew T Meredith, Spring, TX (US); Michael G Duff, Liberty, MO (US)

(21) Appl. No.: 19/205,661

(22) Filed: May 12, 2025

Related U.S. Application Data
(60) Provisional application No. 63/645,634, filed on May 10, 2024.

(51) Int. Cl. A01N 63/20; A01N 25/04; A01N 25/22; A01N 25/30; A01N 63/30; A01P 21/00; C12N 1/14; C12N 1/20

(52) U.S. Cl. CPC A01N 63/20; A01N 25/04; A01N 25/22; A01N 25/30; A01N 63/30; A01P 21/00; C12N 1/14; C12N 1/20

(57) ABSTRACT

An agricultural formulation including a suspension of one or more sporulated bacteria or fungi, a concentrated sugar solution, one or more surfactants, and one or more rheology modifiers.

### TECHNICAL FIELD

This disclosure relates to the field of liquid microbial formulations. Specifically, the present disclosure relates to fungal or bacterial spores that are formulated into a concentrated, low-viscosity suspension where they do not agglomerate together or grow (de-sporulate), and where they stay viable and dormant until use.

### BACKGROUND

A host of bacterial and fungal species have been discovered that are beneficial to a range of application areas such as bioremediation, water treatment, and plant growth. In bioremediation, bacteria and fungi can digest/decompose toxic or persistent chemicals present in soil or water more efficiently than chemical extraction methods. In agricultural applications, some bacteria and fungi can function as biopesticides, which help control plant pathogens or detrimental fungi, insects, mites, or nematodes through their exudates or by directly feeding on the target pest. Plant-beneficial bacteria and fungi can also provide non-pesticidal bio stimulation, which improves plant health and growth characteristics. Endophytic bacteria are those which inhabit and colonize plant cells, similar to the way that certain beneficial bacteria inhabit the intestines of mammals. These endophytes spend part of their life cycle in a symbiotic relationship with their hosts, providing them with a multitude of benefits, such as increased nutrient uptake and availability, resistance to pests and diseases, and increased tolerance to abiotic stressors. Another class of beneficial bacteria are rhizobacteria which live in the rhizosphere—the area of soil immediately surrounding the plant roots. Rhizobacteria can be beneficial to plants in a number of ways, such as releasing biostimulating compounds, assimilating atmospheric nitrogen into a usable form (nitrogen fixing), promoting nutrient uptake, suppressing the growth of plant pathogens, and improving plant stress tolerance. Many endophytes are also rhizobacteria and spend their life cycling between the soil and the interior of plant roots.

All of these plant-beneficial bacteria and fungi have many benefits over traditional pesticides and fertilizers in promoting plant growth—they are more environmentally friendly, sustainably produced without the use of petrochemicals, they may be more effective and more specific in controlling pests that have developed resistance to conventional pesticides, they reproduce in the soil throughout the growing cycle, and they are often specific in their function, which reduces un-desired harm to beneficial organisms. These types of bacteria and fungi have the potential to supplement, replace, or synergistically add to the use of synthetic fertilizers, which are known to cause some environmental issues such as eutrophication and waterway pollution.

From a practical standpoint, however, living organisms can be more difficult than traditional agrochemicals to formulate and store in a stable, liquid, concentrated form that is easy for the applicator to use. Microbes are relatively large on a molecular scale and are not able to be solubilized/dissolved into a solvent or water, like many small molecules, and so must be formulated as a suspension. To form a shelf-stable suspension product, regardless of chemistry, the particles in the formulation must stay suspended in solution, must be prevented from agglomerating, and must maintain chemical stability. In biological formulations described above, additional challenges are present: The organisms must remain in a dormant form during storage and then “wake up” at the right time, after they are delivered to the target plants. If the organisms wake up early and multiply in the storage container, they can cause unwanted growth, off-gassing and pressurization, or release undesirable chemicals into the formulation. On the other hand, they can also lose viability during storage if the microbes and fungi degrade or react chemically with their co-formulants, have their membranes disrupted by solvents or certain surfactants, or become ruptured due to ice crystals, each of which results in cell death.

One way to improve shelf life and effectiveness of these formulations is to use bacteria or fungi in their sporulated forms. Spores are dormant forms of bacteria or fungi which allow an organism to survive harsh environmental conditions such as heat, cold, or desiccation, and can allow for these organisms to survive for many years. Then, when environmental conditions become favorable for growth, bacterial or fungal spores can germinate and grow into new vegetative cells that reproduce and carry out their desired function. The germination process is triggered by a variety of factors, including moisture levels, nutrients, and temperature.

While sporulated microbes are more stable and resistant to degradation by environmental factors, their formulation presents unique challenges. Spores must be formulated in an environment that prevents them from germinating and beginning to grow. They also must be formulated as a stable suspension with minimal settling. Settling could result in an uneven distribution of product in the container that causes mis-dosing of the spores. Microbial spores have a hydrophobic outer layer that can be composed of chemically resistant sporopollenins or other hydrophobic proteinaccous compounds. This hydrophobic outer layer causes microbes to aggregate when dispersed in most aqueous solutions, and this agglomeration leads to settling, and should be avoided.

The many pitfalls of formulating microbes and/or sporulated microbes has led to the commercialization of products that are difficult to handle. Dry powders are typically dusty, hard to measure, tend to clump together due to the hygroscopic nature of spray-dried microbial broth, and as such are undesirable to handle in large quantities in the field. Many liquid biological formulations have special handling conditions that must be adhered to, such as narrow temperature windows and short shelf lives.

Because of this, various strategies for the formulation of sporulated microbes have been explored but no single formulation is able to stabilize all spores due to differences in chemistry and physical properties. There is a need for additional stable formulations of microbial spores.

High concentrations of sugar are known to prevent spores from germinating and can actually have a biocidal and/or biostatic effect (for example products like jams, jellies, honey, etc. have long shelf lives at room temperature) due to the high osmotic pressure that exists in low-water sugar solutions. However, these sugar concentrates do not contain any physical stabilizing properties necessary to keep spores suspended, and many times at the concentration required to form a biostatic environment have a viscosity too high for easy handling, so formulation aids would be necessary to make functional products with a reasonable shelf life. Many typical formulation aids (dispersants, surfactants) that are used to formulate agrochemical active ingredients are not compatible with low-water solutions such as highly concentrated sugars, so it is not intuitive to one skilled in the art as to which formulation aides will be effective at physically stabilizing spores in these solutions. Lewis in U.S. Pat. No. 2,402,902 formulates ergot spores into sugar solutions to prevent germination but does not provide any means for stabilizing the suspensions, which would result in a short shelf life and packing of the spores at the bottom of the container. Other descriptions of systems and techniques related to those described herein may include US Patents and Patent Applications US20220211046A1—liquid bacterial spore formulations; US20210127684A1—bacillus strains and microfibrillated cellulose; U.S. Pat. No. 10,362,786—spore formulations in glycerin; U.S. Pat. No. 10,383,339B2—biopesticide spores with sorbitan surfactants; US20230337681A1—microbial suspensions in oils; U.S. Pat. No. 2,402,902—formulates ergot spores into sugar solutions to prevent germination; each of which is incorporated by reference herein in its entirety.

### SUMMARY

We have now surprisingly found that combinations of concentrated sugar solutions with dispersants, surfactants, viscosity-reducing hydrogen bonded complexes, and rheology modifiers are capable of stabilizing suspensions of sporulated microbes for long periods of time, keeping the spores viable and suspended in solution without significant settling or microbial degradation.

In some examples, the disclosure describes a formulation including a suspension of one or more species of sporulated bacteria or fungi, a concentrated sugar solution, one or more surfactants, and one or more rheology modifiers. The formulation may contain less than 50% water, such as less than 40% water. The total amount of sporulated bacteria or fungi may be in the range of 0.1% to 25% by weight. The total colony forming units per gram of material may be in the range of 1×10⁵ to 1×10¹². The one or more surfactants may have an HLB between 7 and 15. The one or more surfactants may include at least one or an esterified sorbitan ethoxylate, alkyl polyglucoside, polyethylene glycol fatty acid ester, and alkoxylated C6-C24 aliphatic alcohol. The one or more rheology modifiers may impart shear-thinning properties to the formulation, imparts thixotropic properties to the formulation, include a hydrated gum comprising xanthan gum, guar gum, tamarind gum, or synthetically modified gum, include a microfibrillated cellulose, and/or include a modified clay. The formulation may further include a compound that forms a hydrogen bonded complex with the sugar and lowers the viscosity of the formulation. Additionally, the formulation may include one or more of a nitrogen fertilizer, an alkylene glycol or glycerin co-solvent, a dispersant, and at least one of a humic substance, humic acid, and fulvic acid.

In some examples, the disclosure describes a method for stabilizing at least one of a sporulated bacteria and fungi formulation. The method includes dispersing the at least one sporulated bacteria and fungi in a concentrated sugar solution that contains dissolved surfactants, dispersants, and one or more rheology modifiers.

In some examples, the disclosure describes a method of treating a plant or plant seed. The method includes diluting a formulation including a suspension of one or more species of sporulated bacteria or fungi, a concentrated sugar solution, one or more surfactants, and one or more rheology modifiers into a sprayable or injectable solution. The method also includes at least one of spraying the diluted formulation onto a plant or soil around a plant and injecting the diluted formulation in the root zone of a germinating or soon-to-germinate seed.

### DESCRIPTION OF THE DRAWINGS

The following figures illustrate exemplary subject matter described in this disclosure. herein.

FIG. 1 is graph illustrating data from Example 2.

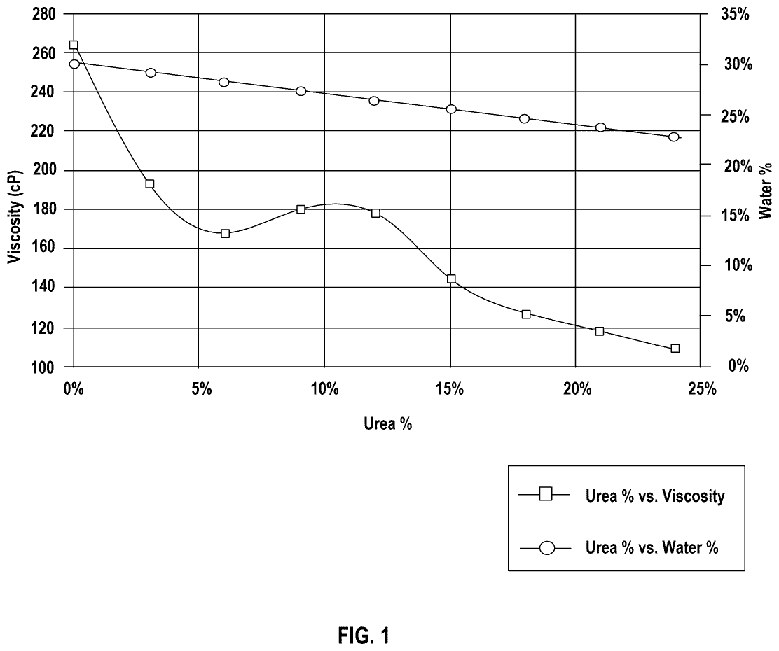

FIG. 2 is a photograph of HFCS+urea samples from Example 2, the left-most sample is 0% urea, and the right-most sample is 24% urea.

FIG. 3 is a photograph of the formulation of Example 4 on the left, and a photograph of the formulation of Comparative example 1 on right, after 6 weeks of accelerated aging.

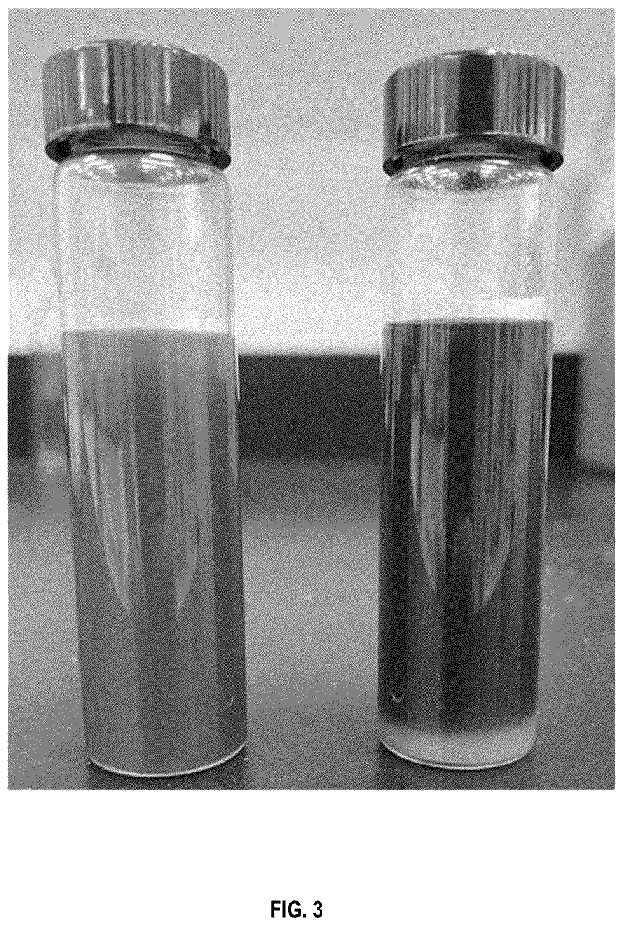

FIG. 4 is a 400× microscope image of Example 4 formulation showing well-dispersed spores.

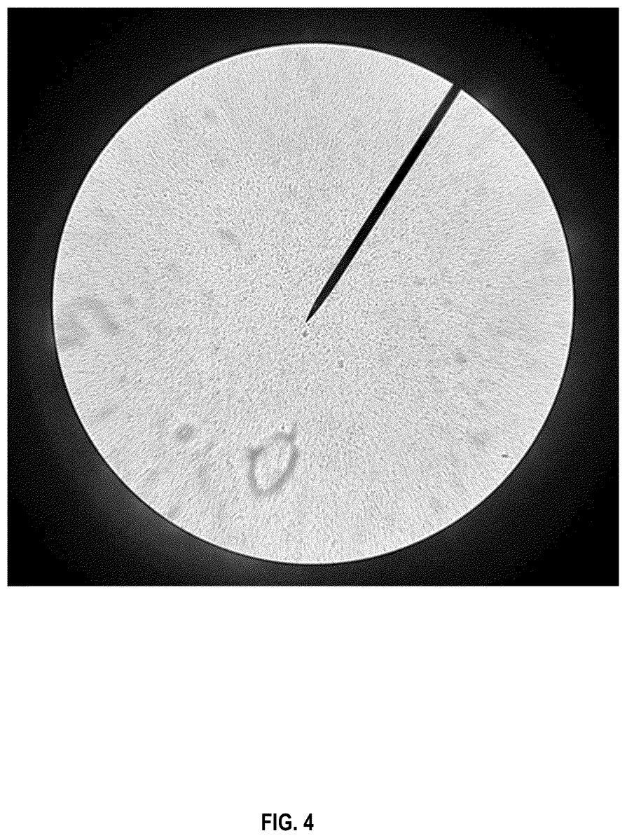

FIG. 5 is a 400× microscope image of Comparative example 2 formulation showing agglomerated spores.

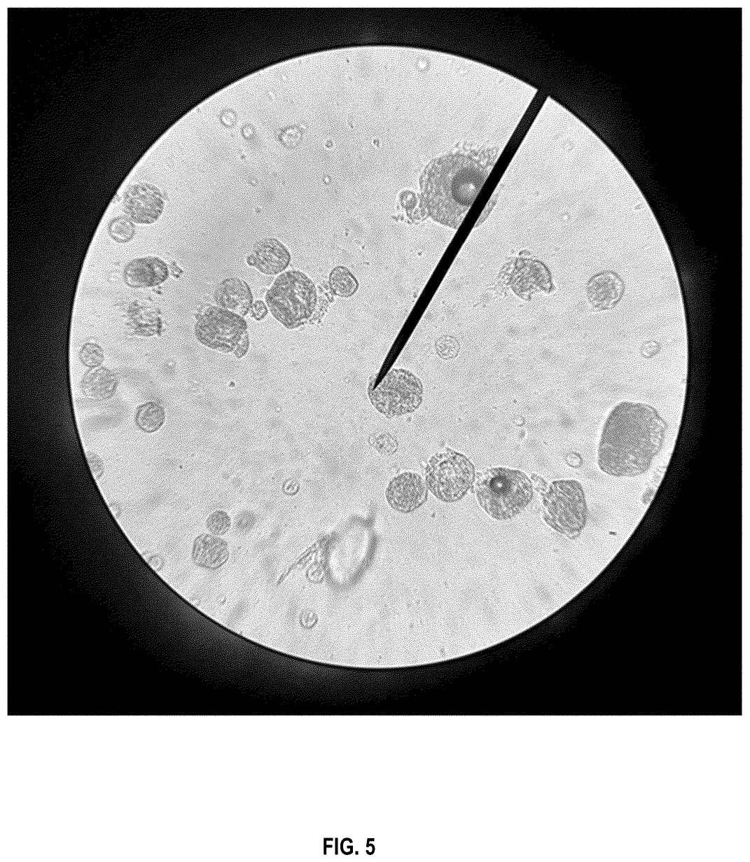

FIG. 6 is a photograph of the formulation of Example 4 (left) and the formulation of Example 5 (right) after 2 years of storage at 2° C.

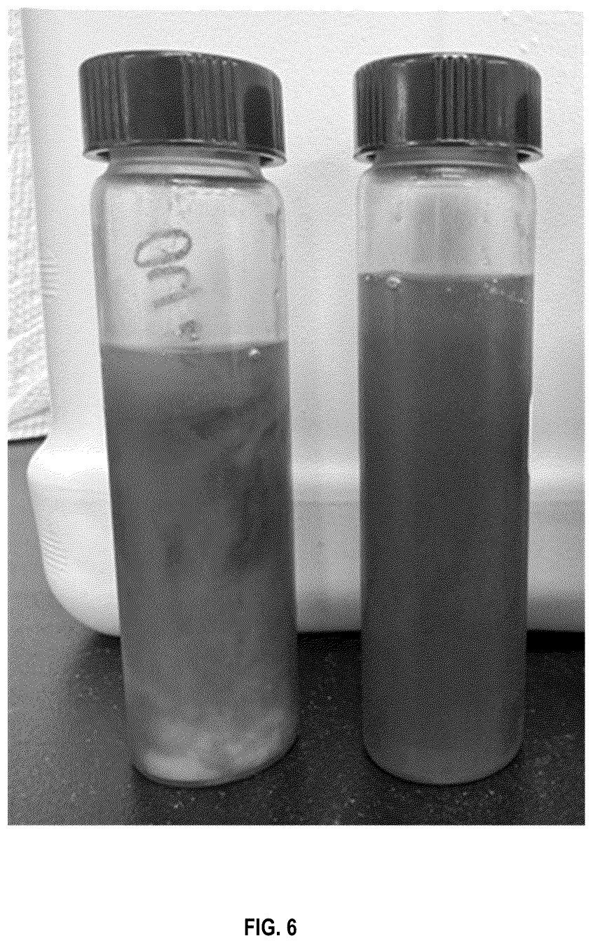

FIG. 7 is a photograph of corn plants at intermediate stage of growth showing enhanced root development after application of the formulation from Example 5 (left) vs. no microbial formulation (right).

FIG. 8 is a photograph of soybean plants at intermediate stage of growth showing enhanced root development after application of formulation from Example 5 (left) vs. no microbial formulation (right).

### DETAILED DESCRIPTION

For purposes of promoting an understanding of the principles of the disclosure, reference will now be made to the embodiments illustrated in the drawings, and specific language will be used to describe the same. It will nonetheless be understood that no limitation of the scope of the disclosure is intended by the illustration and description of certain embodiments of the disclosure. In addition, any alterations and/or modifications of the illustrated and/or described embodiment(s) are contemplated as being within the scope of the present disclosure. Further, any other applications of the principles of the disclosure, as illustrated and/or described herein, as would normally occur to one skilled in the art to which the disclosure pertains, are contemplated as being within the scope of the present disclosure.

The present disclosure describes agricultural formulations having combinations of concentrated sugar solutions with dispersants, surfactants, hydrogen bonded complexes, and rheology modifiers configured to stabilize suspensions of sporulated microbes for long periods of time, keeping the spores viable and suspended in solution without significant settling or microbial degradation. The described formulations typically comprise 40-70% of a concentrated sugar solution such as high fructose corn syrup, corn syrup, sucrose, fructose, glucose, etc., 0.1-20% of a sporulated microbial concentrate, 1-10% of one or more wetting and/or emulsifying surfactants, 0.1-5% of one or more dispersants, one or more glycol cosolvents, one or more rheology modifiers such as xanthan gum, guar gum, hydroxypropyl guar gum, carboxymethyl cellulose, sodium alginate, pectin, locust bean gum, tamarind gum, modified tamarind gum, fumed silica, microfibrillated cellulose, nanoclays, or polyacrylates, optionally one or more compounds that form a hydrogenbonded network with the sugar such as urea, optionally other nitrogen sources, and optionally one or more biostimulant compounds. The formulations contain less than 50% total water, and more preferably, less than 40% water.

Sugar solutions may be obtained as commercial mixtures such as various grades of corn syrup, high fructose corn syrup, etc. or may be mixed by adding solid sugars to water, such as sucrose, glucose, fructose, xylose, etc.

The sporulated microbe powder is obtained by growing the desired bacteria and/or fungi in a culture medium to the desired concentration of living cells, inducing sporulation by withholding key nutrients (carbon, nitrogen, phosphorus, etc.), concentration of the spores through centrifugation or filtering, drying, and homogenizing through a milling, blending, or mixing process. Microbial spores used in the invention can be obtained from any spore-forming bacterial or fungal genera, including, but not limited to, Bacillus, Streptomyces, Thermobacillus, Thiobacillus, Pseudomonas, Rhodopseudomonas, Trichoderma, and Frankia.

Surfactants in the formulation can be from any number of classes of surfactants, including, but not limited to, alcohol alkoxylates, ether sulfates, alkylbenzene sulfonates, alkyl polyglucosides, esterified sorbitan ethoxylates, sorbitan esters, alkylamine alkoxylates, and betaines.

Dispersants in the formulation can be from any number of synthetic or natural sources, including, but not limited to, polyacrylates, PEGylated polyacrylates, polyamines, styrene methacrylate copolymers, modified styrene-maleic anhydride co-polymers, lignosulfonates, naphthalene sulfonates, alkylnaphthalene sulfonates, alkylnaphthalene sulfonate formaldehyde condensates, and tristyrylphenol alkoxylate sulfates or phosphates.

Solvents and/or co-solvents in the formulation can include any suitable solvent, including, but not limited to, glycols, polyalkylene glycols, glycol ethers, amides (N-methylpyrrolidone, dimethylformamide, dimethylacetamide), DMSO, alcohols, ethers, or ketones.

The rheology modifiers are typically polymers which, when added to the formulation, give them a shear-thinning property, which enhances storage stability. Rheology modifiers can include any suitable rheology modifier, including, but not limited to, xanthan gum, guar gum, tamarind gum, fumed silica, nanocelluloses, modified celluloses, microfibrillated cellulose, organically modified clays, polyethylene oxide, or polyvinyl pyrrolidone.

Optional compounds that reduce the viscosity of the sugar solution through hydrogen bonding complex formations with the sugar include urea, pyridine, aliphatic tertiary amines, amide solvents, and polyamides.

Optional nitrogen sources in the formulation include, but are not limited to, amines, ethanolamines, propanolamines, urea, hydroxyethylurea, alkylureas, tetramethylurea, or amino acids.

Optional biostimulant compounds in the formulation may include, but are not limited to, vitamins, amino acids, growth hormones, humic substances, fulvic acids, seaweed extract, choline, C8-C12 dicarboxylic acids, chitosan, chitin, salicylic acid or its salts, trehalose, and others.

List of formulation components: ETHSORBOX L-20—sorbitol+20EO, monolaurate, produced by Ethox chemical; AltaBio 60—a lignosulfonate dispersant produced by Ingevity corp; Exilva F01-L—a microfibrillated cellulose product produced by Borregard Corp.; SURFONIC TDA-9—a nonionic surfactant produced by Indorama Corp.

### COMPARATIVE EXAMPLE 1

A formulation with the composition shown in Table 1 was mixed in a 600 mL beaker and evaluated for storage stability at room temperature. Within 2 days, the microbial powder sank to the bottom of the container, showing instability.

TABLE 1

| | |
|---|---|
| Water | 14% |
| Microbial spore powder (2 × 10¹¹ CFU/g) | 1.00% |
| Corn Syrup (75% solids) | 85% |

### COMPARATIVE EXAMPLE 2

A high-water formulation with the composition shown in Table 2 was mixed in a 600 mL beaker and evaluated for storage stability at room temperature. While the solids in this formulation stayed suspended, within a week the formulation displayed active microbial growth and pressurization of the storage container due to off gassing of said growth.

TABLE 2

| | |
|---|---|
| Water | 83.60% |
| Microbial spore powder (2 × 1011 CFU/g) | 1.00% |
| Propylene glycol | 6.00% |
| ETHSORBOX L-20 | 3.00% |
| AltaBio 60 | 0.30% |
| 1% xanthan gum pre-mix | 6.00% |
| Silicone antifoam | 0.10% |

### EXAMPLE 1—SURFACTANT SCREEN

Surfactants can help suspend and compatibilize microbial spores in a formulation. A series of nonionic surfactants were screened with the mixture of high fructose corn syrup (HFCS), water, and microbial spore mixture below:

TABLE 3

| Component | % |
|---|---|
| HFCS | 84% |
| Water | 13% |
| Surfactant | 2% |
| Microbial spore | 1% |

TABLE 4

| Surfactant Type | Formulation result |
|---|---|
| Sorbitol + 20EO monolaurate | Homogeneous suspension, slowly settles over 1 week |
| Oleyl alcohol + 4EO, phosphate | Immediate phase separation |
| ester | |
| Castor Oil + 36 EO | Immediate phase separation |
| Tridecyl alcohol + 9EO | Immediate phase separation and microbe agglomeration |
| Sorbitan monooleate | Immediate phase separation |
| Sorbitol + 20EO monooleate | Homogeneous suspension, slowly settles over 1 week |
| Lauryl alcohol + 4 EO, phosphate | Immediate phase separation and microbe agglomeration |
| ester | |
| PEG-600 monooleate | Immediate phase separation and microbe agglomeration |
| N-methyl alkyl glucamide | Homogeneous suspension, slowly settles over 1 week |
| Tristyrylphenol + 16EO, ether | Homogeneous suspension, slowly settles over 1 week |
| sulfate ammonium salt | |
| Sorbitol + 20EO, trioleate | Good microbial suspension but rapid phase separation |
| Lauryl alcohol + 7EO + 7PO | Good microbial suspension but rapid separation |
| Alkyl polyglucoside (APG) | Homogeneous suspension, slowly settles over 1 week |

According to the results in Table 4, there are certain surfactants that evenly disperse/suspend the microbial spores in a concentrated sugar solution, and many that are not compatible with this system. However, even the surfactants that evenly disperse the spores do not keep them suspended over a week because the surfactants do not impart significant viscoelastic properties to the system. Further additives are needed.

### EXAMPLE 2—UREA EFFECT ON HFCS

It was surprisingly found that urea, through a hydrogen bonding complex formed with glucose and/or fructose as outlined in (Carbohydrate Research 12 (1970) 153-156), had a viscosity-lowering effect on the HFCS. HFCS urea mixtures were made by dissolving urea in HFCS, and then viscosity was measured with a Brookfield viscometer. All solutions had Newtonian behavior but surprisingly, as urea content increased, the viscosity dropped. This is the opposite of what one would expect as normally when water content drops in a sugar system, the viscosity increases significantly.

TABLE 5

| Urea % | Water % | Viscosity |
|---|---|---|
| 0% | 30.00% | 264 |
| 3% | 29.10% | 193 |
| 6% | 28.20% | 168 |
| 9% | 27.30% | 180 |
| 12% | 26.40% | 178 |
| 15% | 25.50% | 145 |
| 18% | 24.60% | 127 |
| 21% | 23.70% | 118 |
| 24% | 22.80% | 109 |

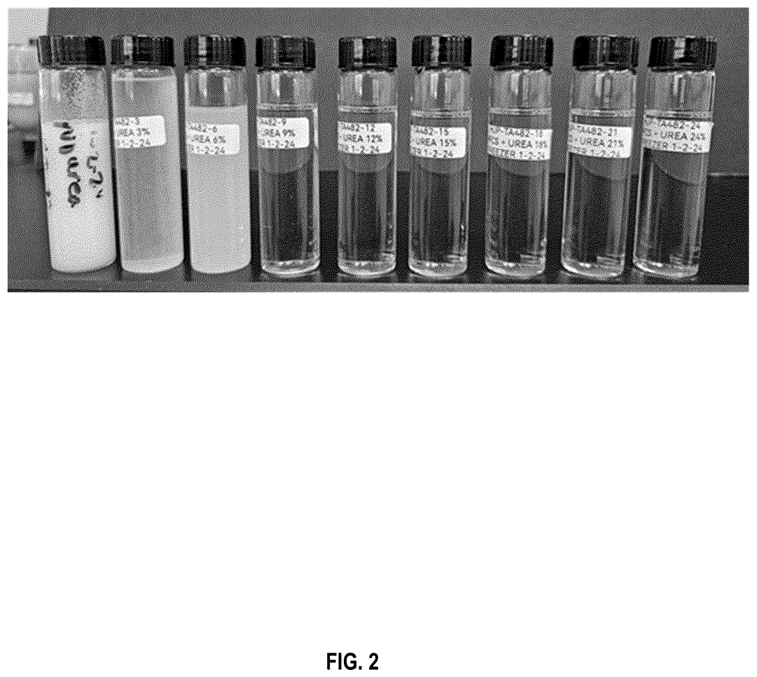

The urea/HFCS solutions were frozen by dropping the solutions to −20° C. and observing over time. After 6 weeks, the samples were warmed to RT and observed. Surprisingly, all solutions above 6% of the urea remained clear, liquid, and homogeneous. This is an advantageous result as it shows that urea can be used to prevent sugar from crystallizing. FIG. 2 shows the samples after freezing and thawing.

### EXAMPLE 3—RHEOLOGY MODIFIER SCREEN

The formulations below were made by dissolving the ingredients using an overhead mixer in a 600 mL beaker, adding the microbial spores, and then once the spores were dispersed adding the specific rheology modifier. The formulation systems were observed under cold storage, RT storage, and warm storage (40° C.). Table 7 lists observations for each.

TABLE 6

| Component | Wt. % |
|---|---|
| HFCS | 56 |
| PG | 8 |
| urea | 11 |
| AltaBio 60 | 0.8 |
| ETHSORBOX L-20 | 3 |
| Rheology modifier | 0.05-2 |
| Microbial spore | 1.0 |
| Water | balance |

TABLE 7

| Rheology modifier | Results/observations |
|---|---|
| Xanthan gum | Compatible, forms shear-thinning and stable suspensions with |
| | high zero-shear viscosity and yield stress |
| Hydroxypropyl guar gum | Compatible, forms shear-thinning suspensions but not enough |
| | yield stress to hold suspension in warm temperatures |
| Native guar gum | Compatible, forms shear-thinning suspensions but not enough |
| | yield stress to hold suspension in warm temperatures |
| Carboxymethyl cellulose | Compatible, forms shear-thinning suspensions but not enough |
| | yield stress to hold suspension in warm temperatures |
| Microfibrillated Cellulose | Compatible, forms shear-thinning suspensions but holds onto air |
| | bubbles and floats to top of formulation over time |
| Polyvinyl alcohol | Not compatible/soluble |
| Polyvinylpyrrolidone | Compatible, forms shear-thinning suspensions but not enough |
| | yield stress to hold suspension in warm temperatures |
| Bentone CT | Not compatible |
| Bentone AD | Not compatible |
| Bentone LT | Compatible but does not impart enough viscosity |
| Bentone DH | Not compatible |
| Bentone EW | Compatible but does not impart enough viscosity |
| Bentone SD-2 | Not compatible |
| Bentone HD | Not compatible |

### EXAMPLE 4

A formulation with the composition below was mixed in a 600 ml beaker and split into vials for evaluation under different storage conditions. The formulation had a viscosity of 600 centipoise (cP) at RT using a Brookfield viscometer, spindle 63 and speed of 30 rpm, and displayed shear-thinning behavior. At 2° C., the viscosity was 1300 cP, and possibly too thick for optimal pumpability in an agrochemical applications system.

TABLE 8

| Component | Wt. % |
|---|---|
| HFCS | 73.60% |
| Microbial spore powder | 1.00% |
| Water | 6.00% |
| Propylene glycol | 6.00% |
| ETHSORBOX L-20 | 3.00% |
| AltaBio 60 | 0.30% |
| 1% xanthan gum pre-mix | 6.00% |
| Exilva F01-L | 4.00% |
| antifoam | 0.10% |

The sample was stored at −20° C., 2° C., 23° C., 40° C., and one sample was cycled between −20° C. and RT every 4 days. After storage for 6 months under these conditions, the high temperature sample displayed about 2% syneresis, and none of the other samples looked different. After 2 years the samples stored at 2° C. had formed large sugar crystals.

The sample was also evaluated after 6 months of storage for microbial viability and no significant change was observed in viable colony forming units (CFUs) per gram.

### EXAMPLE 5

The formulation below was mixed in a 1-gal beaker and evaluated for viscosity and storage stability.

TABLE 9

| Component | Wt. % |
|---|---|
| Component | % |
| HFCS | 56.60% |
| Microbial Spore Product | 1.00% |
| Water | 17.09% |
| Urea | 11% |
| PG | 7.80% |
| ETHSORBOX L-20 | 3.00% |
| AltaBio 60 | 0.80% |
| xanthan gum | 0.13% |
| potassium sorbate | 0.13% |
| Exilva F01-L | 2.40% |
| Antifoam | 0.05% |

This product, due to the addition of the urea, had a lower 2° C. viscosity of 900 cP, and had an RT viscosity of 500 cP. The product was pumpable and usable in an agrochemical applications system. The shelf stability of this formulation is >2 years, with only minimal syneresis forming on top of the containers.

### EXAMPLE 5: FIELD TRIALS

The formulation of Example 4 was made using a phosphate and nitrogen-enhancing microbial spore mixture from New Life Biosciences, Inc. and applied in-furrow to both corn and soy plants along with a liquid fertilizer mixture vs. control applications that did not contain the microbial spore product.

Yield

| Crop | Example 5 @ 4 oz/acre | untreated |
|---|---|---|
| Corn | 227 bu/ac | 219 bu/ac |
| Soy | 63 bu/ac | 55 bu/ac |

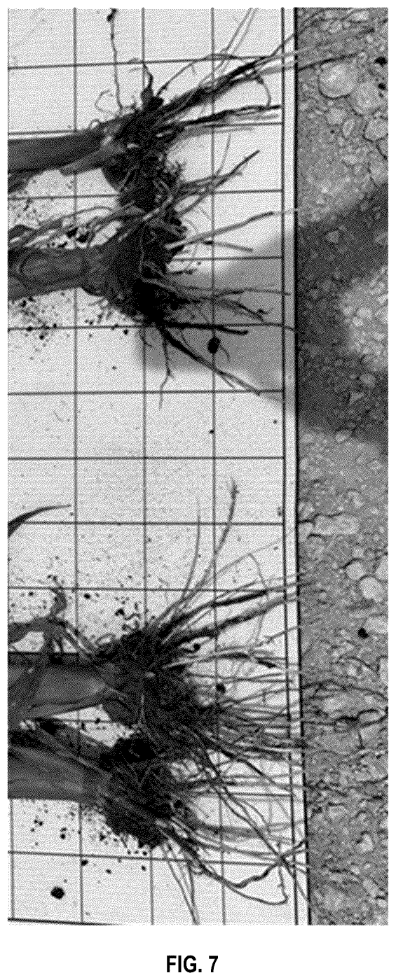

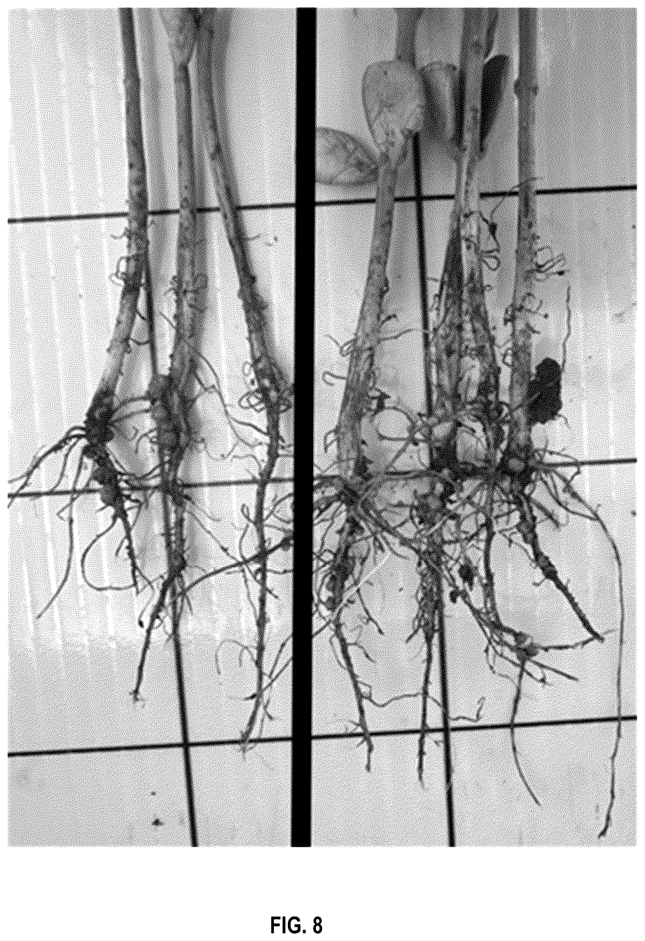

As shown in FIGS. 7-8, root development was significantly enhanced when the microbial spore product was applied.

While the disclosure has been described in connection with what is presently considered to be the most practical and preferred embodiments, it is to be understood that the disclosure is not to be limited to the disclosed embodiments, but on the contrary, is intended to cover various modifications and equivalent arrangements included within the spirit and scope of the appended claims, which scope is to be accorded the broadest interpretation so as to encompass all such modifications and equivalent structures as permitted under the law. Furthermore, it should be understood that while the use of the word preferable, preferably, or preferred in the description above indicates that feature so described may be more desirable, it nonetheless may not be necessary and any embodiment lacking the same may be contemplated as within the scope of the disclosure, that scope being defined by the claims that follow. In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

1. A formulation comprising a suspension of one or more species of sporulated bacteria or fungi, a concentrated sugar solution, one or more surfactants, and one or more rheology modifiers.

2. The formulation of claim 1, containing less than 50% water.

3. The formulation of claim 1, containing less than 40% water.

4. The formulation of claim 1, wherein a total amount of sporulated bacteria or fungi is in the range of 0.1% to 25% by weight.

5. The formulation of claim 1, wherein a total colony forming units per gram of material is in the range of 1×10⁵ to 1×10¹².

6. The formulation of claim 1, wherein the one or more surfactants has a hydrophilic-lipophiulic balance (HLB) value between 7 and 15.

7. The formulation of claim 1, wherein the one or more surfactants is a surfactant including at least one of an esterified sorbitan ethoxylate, alkyl polyglucoside, polyethylene glycol fatty acid ester, and alkoxylated C6-C24 aliphatic alcohol.

8. The formulation of claim 1, wherein the one or more rheology modifiers imparts shear-thinning properties to the formulation.

9. The formulation of claim 1, wherein the one or more rheology modifiers imparts thixotropic properties to the formulation.

10. The formulation of claim 1, wherein the one or more rheology modifiers is a hydrated gum comprising at least one of xanthan gum, guar gum, tamarind gum, and synthetically modified gum.

11. The formulation of claim 1, wherein the one or more rheology modifiers is a microfibrillated cellulose.

12. The formulation of claim 1, wherein the one or more rheology modifiers is a modified clay.

13. The formulation of claim 1, wherein the one or more rheology modifiers is a hydrated gum comprising at least one of a xanthan gum, guar gum, tamarind gum, and synthetically modified gum, and wherein the one or more rheology modifiers configured to impart shear-thinning properties and thixotropic properties to the formulation.

14. The formulation of claim 1, further comprising a compound that forms a hydrogen bonded complex with a sugar of the concentrated sugar solution and lowers a viscosity of the formulation.

15. The formulation of claim 1, further comprising a nitrogen fertilizer.

16. The formulation of claim 1, further comprising an alkylene glycol or glycerin co-solvent.

17. The formulation of claim 1, further comprising a dispersant.

18. The formulation of claim 1, further comprising at least one of a humic substance, humic acid, and fulvic acid.

19. A method for stabilizing at least one of a sporulated bacteria and fungi formulation, wherein the method comprises:
dispersing the at least one sporulated bacteria and fungi in a concentrated sugar solution that contains dissolved surfactants, dispersants, and one or more rheology modifiers.

20. A method of treating a plant or plant seed, wherein the method comprises:
diluting the formulation of claim 1 into a sprayable or injectable solution; and
at least one of spraying the diluted formulation onto a plant or soil around a plant and injecting the diluted formulation in the root zone of a germinating or soon-to-germinate seed.
